(19) United States
(12) Patent Application Publication
(10) Pub. No.: US 2004/0033605 A1
(43) Pub. Date: Feb. 19, 2004

(54) Gene delivery vectors provided with a tissue tropism for dendritic cells

(76) Inventors: Menzo Havenga, Alphen a/d Rijn (NL); Ronald V Vogels, Linschoten (NL); Abraham Bout, Moekapelle (NL)

Correspondence Address: TraskBritt, PO Box 2550, Salt Lake City, UT, 84110, US

(21) Appl. No.: 10/381,088

(22) PCT Filed: Sep. 20, 2001
(86) PCT No.: PCT/EP01/10999

(51) Int. Cl. C12N 15/861; C12N 5/08

(52) U.S. Cl. 435/456; 435/372

(57) ABSTRACT

Adenoviral vectors can be used in vaccines to cause antigen-presenting cells to display desired antigens. Disclosed is a vector and associated means and methods which transduce antigen-presenting cells better than currently available vectors, enabling the vector to be delivered in lower doses, and thus improving the efficiency of adenoviral vaccines technology.

### TECHNICAL FIELD

The present invention relates generally to the field of gene delivery vehicles, particularly gene delivery vehicle having a tissue tropism for dendritic cells, the tissue tropism for dendritic cells being provided by a viral capsid protein.

### BACKGROUND ART

In gene therapy, genetic information is usually delivered to a host cell in order to either correct (supplement) a genetic deficiency in the cell, to inhibit an undesired function in the cell, or to eliminate the host cell altogether. Of course, the genetic information can also be intended to provide the host cell with a desired function, for instance, to supply a secreted protein to treat other cells of the host, etc.

Many different methods have been developed to introduce new genetic information into cells. Although many different systems may work on cell lines cultured in vitro, only the group of viral vector mediated gene delivery methods seems to be able to meet the required efficiency of gene transfer in vivo. Thus, for the purposes of gene therapy, most attention has been directed toward the development of suitable viral vectors, such as vectors based on adenovirus.

Such adenoviral vectors can deliver foreign genetic information very efficiently to target cells in vivo. Moreover, obtaining large amounts of adenovirus vectors are, for most types of adenovirus vectors, not a problem. Adenovirus vectors are relatively easy to concentrate and purify. Moreover, clinical studies have provided valuable information on the use of these vectors in patients.

Many reasons exist for using adenovirus vectors to deliver nucleic acid to target cells in gene therapy protocols. However, some characteristics of the current vectors limit their use in specific applications. For instance, endothelial cells and smooth muscle cells are not easily transduced by the current generation of adenoviral vectors. For many gene therapy applications, these types of cells should be genetically modified. In some applications, however, even the very good in vivo delivery capacity of adenovirus vectors is insufficient, and higher transfer efficiencies are required. This is the case, for instance, when most cells of a target tissue need to be transduced.

Adenoviruses contain a linear double-stranded DNA molecule of approximately 36,000 base pairs (“bp”). This molecule contains identical inverted terminal repeats (“ITRs”) of approximately 90-140 base pairs with the exact length depending on the adenovirus serotype. The viral origins of replication are within the ITRs at the genome ends. The transcription units are divided into early and late regions.

Shortly after infection, the E1A and E1B proteins are expressed and function in transactivation of cellular and adenoviral genes. The early regions E2A and E2B encode proteins (DNA binding protein, pre-terminal protein and polymerase) required for the replication of the adenoviral genome (reviewed in van der Viet, 1995). The early region E4 encodes several proteins with pleiotropic functions, for example, transactivation of the E2 early promoter, facilitating transport and accumulation of viral mRNAs in the late phase of infection and increasing nuclear stability of major late pre-mRNAs (reviewed in Leppard, 1997). The early region 3 encodes proteins that are involved in modulation of the immune response of the host (Wold et al., 1995). The late region is transcribed from one single promoter (major late promoter) and is activated at the onset of DNA replication. Complex splicing and polyadenylation mechanisms give rise to more than 12 RNA species coding for core proteins, capsid proteins (penton, hexon, fiber and associated proteins), viral protease and proteins necessary for the assembly of the capsid and shut-down of host protein translation (Imperiale et al. “Post-transcriptional Control of Adenovirus Gene Expression”, The Molecular Repertoire of Adenoviruses I., pp. 139-171. (W. Doerfler and P. Böhm (editors), Springer-Verlag Berlin Heidelberg 1995).

The interaction of the virus with the host cell has mainly been investigated with the serotype C viruses Ad2 and Ad5. Binding occurs via interaction of the knob region of the protruding fiber with a cellular receptor. The receptor for Ad2 and Ad5 and probably more adenoviruses is known as the “Coxsackievirus and Adenovirus Receptor” or “CAR” protein (Bergelson et al., 1997). Internalization is mediated through interaction of the RGD sequence present in the penton base with cellular integrins (Wickham et al., 1993). This may not be true for all serotypes, for example, serotypes 40 and 41 do not contain a RGD sequence in their penton base sequence (Kidd et al., 1993).

The initial step for successful infection is binding of adenovirus to its target cell, a process mediated through fiber protein. The fiber protein has a trimeric structure (Stouten et al, 1992) with different lengths depending on the virus serotype (Signas et al., 1985; Kidd et al., 1993). Different serotypes have polypeptides with structurally similar N and C termini, but different middle stem regions. The first 30 amino acids at the N terminus are involved in anchoring of the fiber to the penton base (Chroboczek et al., 1995), especially the conserved FNPVYP region in the tail (Arnberg et al, 1997). The C-terminus, or “knob”, is responsible for initial interaction with the cellular adenovirus receptor. After this initial binding, secondary binding between the capsid penton base and cell-surface integrins leads to internalization of viral particles in coated pits and endocytosis (Morgan et al., 1969; Svensson and Persson, 1984; Varga et al., 1991; Greber et al., 1993; Wickham et al, 1993). Integrins are αβ-heterodimers of which at least 14 α-subunits and 8 β-subunits have been identified (Hynes, 1992). The array of integrins expressed in cells is complex and will vary between cell types and cellular environment. Although the knob contains some conserved regions, between serotypes, knob proteins show a high degree of variability, indicating that different adenovirus receptors exist.

At present, six different subgroups of human adenoviruses have been proposed which in total encompass approximately 50 distinct adenovirus serotypes. Besides these human adenoviruses, many animal adenoviruses have been identified (see, e.g., Ishibashi and Yasue, 1984). A serotype is defined on the basis of its immunological distinctiveness as determined by quantitative neutralization with animal antiserum (horse, rabbit). If neutralization shows a certain degree of cross-reaction between two viruses, distinctiveness of serotype is assumed if A) the hemagglutinins are unrelated, as shown by lack of cross-reaction on hemagglutination-inhibition, or B) substantial biophysical/biochemical differences in DNA exist (Francki et al., 1991). The serotypes identified last (42-49) were isolated for the first time from HIV infected patients (Hierholzer et al., 1988; Schnurr et al., 1993). For reasons not well understood, most of such immuno-compromised patients shed adenoviruses that were never isolated from immuno-competent individuals (Hierholzer et al., 1988, 1992; Khoo et al., 1995).

Besides differences towards the sensitivity against neutralizing antibodies of different adenovirus serotypes, adenoviruses in subgroup C such as Ad2 and Ad5 bind to different receptors as compared to adenoviruses from subgroup B such as Ad3, Ad7, Ad11, Ad14, Ad21, Ad34, and Ad35 (see, e.g., Defer et al., 1990; Gall et al., 1996). Likewise, it has been demonstrated that receptor specificity could be altered by exchanging the Ad3 knob protein with the Ad5 knob protein, and vice versa (Krasnykh et al., 1996; Stevenson et al., 1995, 1997). Serotypes 2, 4, 5 and 7 all have a natural affiliation towards lung epithelia and other respiratory tissues. In contrast, serotypes 40 and 41 have a natural affinity for the gastrointestinal tract. These serotypes differ in at least capsid proteins (penton-base, hexon), proteins responsible for cell binding (fiber protein), and proteins involved in adenovirus replication. It is unknown to what extent the capsid proteins determine the differences in tropism found between the serotypes. It may very well be that post-infection mechanisms determine cell type specificity of adenoviruses. It has been shown that adenoviruses from serotypes A (Ad 12 and Ad31), C (Ad2 and Ad5), D (Ad9 and Ad 15), E (Ad4) and F (Ad41) all are able to bind labeled, soluble CAR (sCAR) protein when immobilized on nitrocellulose. Furthermore, binding of these adenovirus serotypes to Ramos cells, that express high levels of CAR but lack integrins (Roelvink et al., 1996), could be efficiently blocked by addition of sCAR to viruses prior to infection (Roelvink et al., 1998). However, the fact that (at least some) members of these subgroups are able to bind CAR does not exclude that these viruses have different infection efficiencies in various cell types. For example, subgroup D serotypes have relatively short fiber shafts compared to subgroup A and C viruses. It has been postulated that the tropism of subgroup D viruses is to a large extent determined by the penton base binding to integrins (Roelvink et al., 1996; Roelvink et al., 1998). Another example is provided by Zabner et al., 1998 who tested 14 different serotypes on infection of human ciliated airway epithelia (“CAB”) and found that serotype 17 (subgroup D) was bound and internalized more efficiently then all other viruses, including other members of subgroup D. Similar experiments using serotypes from subgroup A-F in primary fetal rat cells showed that adenoviruses from subgroup A and B were inefficient whereas viruses from subgroup D were most efficient (Law et al., 1998). Also in this case viruses within one subgroup displayed different efficiencies. The importance of fiber binding for the improved infection of Ad17 in CAF was shown by Armentano et al. (International Patent Appln. WO 98122609) who made a recombinant LacZ Ad2 virus with a fiber gene from Ad17 and showed that the chimaeric virus infected CAE more efficient then LacZ Ad2 viruses with Ad2 fibers.

Thus, despite their shared ability to bind CAR differences in the length of the fiber, knob sequence and other capsid proteins, for example, penton base of the different serotypes may determine the efficiency by which an adenovirus infects a certain target cell. Of interest in this respect is the ability of Ad5 and Ad2 fibers (but not of Ad3 fibers) to bind to fibronectin m and MHC class 1 α2 derived peptides. This suggests that adenoviruses are able to use cellular receptors other than CAR (Hong et al., 1997). Serotypes 40 and 41 (subgroup F) are known to carry two fiber proteins differing in the length of the shaft. The long shafted 4lL fiber is shown to bind CAR whereas the short shafted 4lS is not capable of binding CAR (Roelvink et al., 1998). The receptor for the short fiber is not known.

Most adenoviral gene delivery vectors currently used in gene therapy are derived from the serotype C adenoviruses Ad2 or Ad5. The vectors have a deletion in the E1 region, where novel genetic information can be introduced. The E1 deletion renders the recombinant virus replication defective. It has been demonstrated extensively that recombinant adenovirus, in particular serotype 5 is suitable for efficient transfer of genes in vivo to the liver, the airway epithelium and solid tumors in animal models and human xenografts in immuno-deficient mice (Bout 1996, 1997; Blaese et al., 1995).

Gene transfer vectors derived from adenoviruses (adenoviral vectors) have a number of features that make them particularly useful for gene transfer:

1) the biology of the adenoviruses is well characterized,

2) the adenovirus is not associated with severe human pathology,

3) the virus is extremely efficient in introducing its DNA into the host cell,

4) the virus can infect a wide variety of cells and has a broad host-range,

5) the virus can be produced at high titers in large quantities, and

6) the virus can be rendered replication defective by deletion of the early-region 1 (E1) of the viral genome (Brody and Crystal, 1994).

However, a number of drawbacks are still associated with the use of adenoviral vectors:

1) Adenoviruses, especially the well investigated serotypes Ad2 and Ad5, usually elicit an immune response by the host into which they are introduced,

2) it is currently not feasible to target the virus to certain cells and tissues,

3) the replication and other functions of the adenovirus are not always very well suited for the cells, which are to be provided with the additional genetic material, and

4) the serotypes Ad2 or Ad5, are not ideally suited for delivering additional genetic material to organs other than the liver.

The liver can be particularly well transduced with vectors derived from Ad2 or Ad5. Delivery of such vectors via the bloodstream leads to a significant delivery of the vectors to the cells of the liver. In therapies where other cell types than liver cells need to be transduced, some means of liver exclusion must be applied to prevent uptake of the vector by these cells. Current methods rely on the physical separation of the vector from the liver cells, most of these methods rely on localizing the vector and/or the target organ via surgery, balloon angioplasty or direct injection into an organ via for instance needles. Liver exclusion is also being practiced through delivery of the vector to compartments in the body that are essentially isolated from the bloodstream thereby preventing transport of the vector to the liver. Although these methods mostly succeed in avoiding gross delivery of the vector to the liver, most of the methods are crude and still have considerable leakage and/or have poor target tissue penetration characteristics. In some cases, inadvertent delivery of the vector to liver cells can be toxic to the patient. For instance, delivery of a herpes simplex virus (“HSV”) thymidine kinase (“TK”) gene for the subsequent killing of dividing cancer cells through administration of gancyclovir is quite dangerous when also a significant amount of liver cells are transduced by the vector. Significant delivery and subsequent expression of the HSV-TK gene to liver cells is associated with severe toxicity.

Dendritic cells are antigen presenting cells (“APC”), specialized to initiate a primary immune response. They are also able to boost a memory type of immune response. Dependent on their stage of development, dendritic cells display different functions: immature dendritic cells are very efficient in the uptake and processing of antigens for presentation by Major Histocompatibility Complex (“MHC”) class I and class II molecules, whereas mature dendritic cells, being less effective in antigen capture and processing, perform much better at stimulating naive and memory CD4⁺ and CD8⁺ T cells, due to the high expression of MHC molecules and co-stimulatory molecules at their cell surface. The immature DCs mature in vivo after uptake of antigen, travel to the T-cell areas in the lymphoid organs, and prime T-cell activation.

Since DCs are the cells responsible for triggering an immune response, there has been a long standing interest in loading DCs with immunostimulatory proteins, peptides, or the genes encoding these proteins, to trigger the immune system. The applications for this strategy are in the field of cancer treatment as well as in the field of vaccination. So far, anti-cancer strategies have focused primarily on ex vivo loading of DCs with antigen (protein or peptide). These studies have revealed that this procedure resulted in induction of cytotoxic T cell activity. The antigens used to load the cells are generally identified as being tumor specific. Some, non-limiting, examples of such antigens are GP 100, mage, or Mart-1 for melanoma.

Besides treating cancer, many other potential human diseases are currently being prevented through vaccination. Well-known examples of disease prevention via vaccination strategies include hepatitis A, B, and C, influenza, rabies, yellow fever, and measles. Besides these well-known vaccination programs, research programs for treatment of malaria, ebola, river blindness, HIV and many other diseases are being developed.

Many of the identified pathogens are considered too dangerous for the generation of “crippled” pathogen vaccines. It would thus be an improvement in the art to be able to isolate and characterize proteins of each pathogen to which a “full blown” immune response is mounted, thus resulting in complete protection upon challenge with wild type pathogen.

### DISCLOSURE OF INVENTION

In the herein described vaccination strategy, a “crippled” pathogen is presented to the immune system via the action of the antigen presenting cells, i.e., immature DCs.

According to the invention, adenoviral vectors are used in vaccines to cause antigen-presenting cells to display desired antigens. Disclosed are vectors and associated means and methods which transduce antigen-presenting cells better than currently available vectors, enabling the vector to be delivered in lower doses thus improving the efficiency of the adenoviral vaccine technology.

As is more thoroughly described herein, such a gene delivery vehicle is provided with at least a tissue tropism for dendritic cells. In such a gene delivery vehicle, the tissue tropism for dendritic cells is generally provided by a virus capsid. The virus capsid preferably includes protein fragments derived from at least two different viruses, such as an adenovirus (e.g., an adenovirus of subgroup B, such as a fiber protein derived from a subgroup B adenovirus). An adenovirus capsid with (or provided with) a tissue tropism for dendritic cells may have the capsid comprising proteins from at least two different adenoviruses and at least a tissue tropism determining fragment of a fiber protein is derived from a subgroup B adenovirus.

The cell line PER C6 (IntroGene, by Leiden, N L) can be used to produce vaccines by producing adenoviral vectors that can safely deliver a portion of a pathogen's DNA into the body, provoking an immune response against the disease.

The invention also includes pharmaceutical compositions, such as vaccines, that include the gene delivery vehicle of the invention and use of the composition to treat or prevent disease.

### BRIEF DESCRIPTION OF THE DRAWINGS

FIG. 1: Transduction of immature dendritic cells (“DCs”) at a virus dose of 100 or 1000 virus particles per cell. Virus tested is Ad5 and Ad5 based vectors carrying the fiber of serotype 12 (Ad5.Fib12), 16 (Ad5.Fib16), 28 (Ad5.Fib28), 32 (Ad5.Fib32), the long fiber of 40 (Ad5.Fib40-L, 49 (Ad5.Fib49), 51 (Ad5.Fib51). Luciferase transgene expression is expressed as relative light units per microgram of protein.

FIG. 2: Flow cytometric analyses of LacZ expression on immature and mature DCs transduced with 10000 virus particles per cell of Ad5 or the fiber chimeric vectors Ad5.Fib 16, Ad5.Fib40-L, or Ad5.Fib51. Percentages of cells scored positive are shown in the upper right corner of each histogram.

FIG. 3: Luciferase transgene expression in human immature DCs measured 48 hours after transduction with 1000 or 5000 virus particles per cell. Viruses tested were fiber chimeric viruses carrying the fiber of subgroup B members (serotypes 11, 16, 35, and 51).

FIG. 4: GFP expression in immature human DCs48 hours after transduction with 1000 virus particles per cell of Ad5, Ad5.Fib16, and Ad5.Fib35. Non-transduced cells were used to set a background level of approximately 1% (−).

FIG. 5: Transduction of mouse and chimpanzee DCs. Luciferase transgene expression measured in mouse DCs 48 hours after transduction is expressed as relative light units per microgram of protein. Chimpanzee DCs were measured 48 hours after transduction using a flow cytometer. GFP expression demonstrates the poor transduction of Ad (35) in contrast to Ad5.Fib35 (66%).

FIG. 6 is a graph charting relative light units (“RLU”) per 10⁴ DC for various recombinant fiber modified vectors.

FIG. 7 consists of two graphs charting GFP expression determined 24 hours after virus exposure. The results are expressed as (a) percentage GFP positive cells and (b) median fluorescence intensity. Dosages used: 10³, 10⁴, or 10⁵ virus particles per DC (white bar, grey bar, or black bar respectively).

FIG. 8 consists of two graphs (a and b) comparing (a) percentage GFP positive cells and (b) median fluorescence intensity after immature DCs were treated with LPS to allow maturation of the DC. Matured DCs were incubated with Ad5. GFP or Ad5Fib 16. GFP, Ad5Fib35.GFP, Ad5fib40-L GFP, or Ad4.Fib51.GFP. Dosages used were: 10³, 10⁴, or 10⁵ virus particles per DC (white bar, grey bar, or black bar respectively).

FIG. 9 is a bar graph showing % GFP+DC (percentage GFP positive cells) for various recombinant fiber-modified vectors. The maturation agents used were LPS (black bars), TNF-a (white with black dots), MCM (diagonal downward), poly I:C (black with white dots), and anti-CD40 antibodies (diagonal upwards). As a negative control for a maturation marker, IFN-a (grey bars) was used. Immature DC s were used a control (white bars).

FIG. 10 consists of three bar graphs (a, b, and c). The DC types were immature DC (white bar), mature DC (black bar), or immature DC (grey bar) transduced and subsequently matured using LPS. Shown are (a) percentage GFP positive cells detected, (b) median fluorescence intensity, and (c) cells that were frozen and the genomic DNA extracted to quantify the number of adenoviral genomes using real-time PCR.

FIG. 11 consists of three graphs comparing IFN-gamma production by immature DCs transduced with 10⁵, 10⁴ or 10³ virus particles (top, middle, and bottom, respectively) of Ad5.gp100 or Ad5.Fib35.gp100 (white squares and circles, respectively). Likewise, matured DC were transduced with 10⁵, 10⁴ or 10³ virus particles of Ad5.gp100 or Ad5.Fib35.gp100 are depicted (black squares and circles, respectively).

FIG. 12: Smooth cells derived from the carotid artery of either human, rhesus, rabbit, rat, mouse, or pig origin, were seeded simultaneously. The cell concentration was 10⁶ cells per well of 24-well plates. Twenty-four hours later, cells were exposed for two hours to a concentration of 1000, 5000, or 10000 virus particles per cell of Ad5 or Ad5Fib 16 carrying luciferase. Cells were infected with virus originating from a single batch of diluted virus. Forty-eight hours later cells were lysed and luciferase activity was determined as described previously. Luciferase activity is described as relative light units (RLU) per microgram cellular protein.

FIG. 13: Dendritic cells from blood originating from cynomolgus, rhesus, or chimpanzees were tested for their sensitivity towards different adenoviral vectors, i.e., Ad5, Ad5Fib16, Ad5Fib35, and Ad5Fib50. Hereto, cells were exposed to 1000 virus particles per cells of each of these vectors carrying GFP. To determine the percentage of cells positive for GFP a flow cytometer was used. To set the flow cytometric background, non-transduced cells were taken (background set at 1%).

FIG. 14: Arrows indicate the position of different subpopulations of cells present in PBMC after gating on the monocytes and lymphocyte (top FIG.) and staining with CD33-PerCP/Cy5 and CD14/CD16-PE (lower FIG.).

FIG. 15: Peripheral blood cells were exposed to 100 vp/cell of either Ad5 or Ad5Fib35 carrying GFP. Twenty-four hours after virus exposure, cells were stained with CD14, CD16 and CD33 to visualize different hemopoietic lineages. Non-transduced cells were used to set the flow cytometric gates at a background level of 1% or less (vertical line). Percentages of cells scored positive are indicated in the upper right corner of each histogram.

FIG. 16: Peripheral blood lymphocytes were exposed to 0, 30, 60 or 100 vp/cell of Ad5, Ad5Fib16 or Ad5Fib35 carrying GFP. Infection was allowed for 2 hours, cells were washed and after twenty-four hours, cells were stained with CD14, CD16 and CD33 to visualize different hemopoietic lineages using flow cytometry. Indicated is the mean GFP-fluorescence of each sub-population, transduced with the different viruses.

FIG. 17: Identification of CD11c⁺ (myeloid) and CD11c⁻ (lymphoid) DC in human blood.

FIG. 18: Analyses of cells expressing GFP that are simultaneously positive (Myeloid) or negative (lymphoid) for membrane marker CD11c. GFP expression is expressed in mean GFP fluorescence.

FIG. 19: Principles of the Elispot assay. Wells of 96-well plates (Millipore, MAHA-S4510) are coated with rat-anti-mouse interferon-gamma antibodies (Pharmingen, Cat no. 18112D) at a concentration of 0.5 mg/ml (100 microliter per well). After 24 hours at 4 degrees Celsius, excess antibody is removed by washing with PBS and wells are filled with 100 microliter of Iscoves medium supplemented with 10% FCS (Life Technologies). Plates are incubated for 1 hour at 37° C. prior to the addition of both target cells (cells containing the proper haplotype and expressing the epitope of interest, in this case gp100) and effector cells, i.e. the 8J gp100 specific CTL (ratio between effector and target cell may be varied). Interferon-gamma produced by activated T-cells are captured by the anti-interferon-gamma antibodies and cells are removed from the wells (time to allow interferon-gamma production to take place maybe varied). Next, antibodies (1/200 diluted in PBS) containing an alkaline phosphatase group are added to the wells (Sigma E-2636) and are allowed to bind at 4 degrees Celsius for 24 hours. Finally 100 microliter substrate is added (1/2000 diluted) and the coloring reaction is stopped by the addition of 100 microliter tap water. The substrate solution is 5-bromo-4-chloro-3-indolyl-phosphate-nitro-blue-tetrazolium (Sigma B5655) and is simply prepared by dissolving one tablet in 10 ml of milliQ water. As an example, a positive and negative well is shown in the right lower corner).

FIG. 20: Detection of interferon-gamma production by CTL clone 8J after stimulation with sorted cells infected with 1000 virus particles per cell of Ad5Fib35 carrying gp100. Upper right panel: sorted cells representing lymphocyte, monocytes, natural killer cells (NK) dendritic cells (CD11c+ and CD11c−) and total PBMCs were subjected to the interferon-gamma test, clearly showing the presence of activated T-cells in the wells containing dendritic cells and, to a much lesser extend, monocytes only. Upper left panel: Identical to upper right panel except that no virus was added to the sorted cell fractions. Lower panel: positive controls using monocyte derived dendritic cells infected with Ad5Fib35-gp100 at a vector dosage of 1000 virus particles per cell. In this panel of positive controls negative controls are taken along by not adding the gp100 CTL clone (−8J).

### BEST MODE OR MODES FOR CARRYING OUT THE INVENTION

A gene delivery vehicle according to the invention preferably has at least one of the protein fragments comprising a tissue tropism determining fragment of a fiber protein derived from a subgroup B adenovirus. Preferably, at least one of the protein fragments comprises a tissue tropism determining fragment of a fiber protein derived from a subgroup B adenovirus. A still more preferred gene delivery vehicle has at least one of the protein fragments comprising a tissue tropism determining fragment of a fiber protein derived from a subgroup B adenovirus, such as adenovirus 16.

In one embodiment, however, a gene delivery vehicle according to the invention, further includes protein fragments derived from an adenovirus of subgroup C.

Also, the gene delivery vehicle can include a nucleic acid derived from one or more adenovirus.

In one embodiment, the gene delivery vehicle according to the invention, has a nucleic acid comprising at least one sequence encoding a fiber protein comprising at least a tissue tropism determining fragment of a subgroup B adenovirus fiber protein, preferably of adenovirus 16.

Furthermore, the adenovirus nucleic acid can be modified such that the capacity of the adenoviral nucleic acid to replicate in a target cell has been reduced or disabled or the adenoviral nucleic acid can be modified so that the capacity of a host immune system to mount an immune response against adenoviral proteins encoded by the adenovirus nucleic acid has been reduced or disabled.

A gene delivery vehicle according to the invention can comprise a minimal adenovirus vector or an Ad/AAV chimaeric vector and can comprise at least one non-adenovirus nucleic acid.

To load DCs with immunostimulatory proteins or peptides to become therapeutically feasible at least two distinct criteria have to be met. First, the isolation of large numbers of DCs that can be isolated, manipulated, and re-infused into a patient, making the procedure autologous. To date, it is possible to obtain such large quantities of immature DCs from cultured peripheral blood monocytes from any given donor. Second, a vector that can transduce DCs efficiently such that the DNA encoding for an immunostimulatory protein can be delivered. The latter is extremely important since it has become clear that the time required for DCs to travel to the lymphoid organs is such that most proteins or peptides are already released from the DCs, resulting in incomplete immune priming. Because DCs are terminally differentiated and thus non-dividing cells, recombinant adenoviral vectors are being considered for delivering the DNA encoding for antigens to DCs. Ideally, this adenovirus should have a high affinity for dendritic cells, but should also not be recognized by neutralizing antibodies of the host such that in vivo transduction of DCs can be accomplished. The latter would obviate the need for ex vivo manipulations of DCs but would result in a medical procedure identical to the vaccination programs that are currently in place, i.e., intramuscular or subcutaneous injection predominantly. Thus, dendritic cells transduced by adenoviral vectors encoding an immunogenic protein may be ideally suited to serve as natural adjuvants for immunotherapy and vaccination.

Efficient gene delivery to DCs is a major interest in the field of gene therapy. Therefore, alteration of the Ad5 host cell range to be able to target DCs in vitro as well as in vivo is a major interest of the invention. To identify a chimeric adenovirus with preferred infection characteristics for human DCs, we generated a library of Ad5 based viruses carrying the fiber molecule from alternative serotypes (serotypes 8, 9, 13, 16, 17, 32, 35, 45, 40-L, 51). Ad5 was included as a reference.

As more thoroughly herein, the susceptibility of human monocyte derived immature and mature dendritic cells to recombinant chimeric adenoviruses expressing different fibers was evaluated.

The invention is further explained by the use of the following illustrative examples:

### EXAMPLES

### Example I

An Ad5/fiber35 Chimeric Vector with Cell Type Specificity for Dendritic Cells

Human PBMC from healthy donors were isolated through Ficoll-Hypaque density centrifugation. Monocytes were isolated from PBMC by enrichment for CD14⁺ cells using staining with FITC labeled anti-human CD 14 monoclonal antibody (Becton Dickinson), anti-FITC microbeads, and MACS separation columns (Miltenyi Biotec).

This procedure usually results in a population of cells that are <90% CD14⁺ as analyzed by FACS. Cells were placed in culture using RPMI-1640 medium (Gibco) containing 10% Foetal Bovine Serum (“FBS”) (Gibco), 200 ng/ml rhu GM-CSF (R&D/ITK diagnostics, 100 ng/ml rhu IL-4 (R&D/ITK diagnostics) and cultured for 7 days with feeding of the cultures with fresh medium containing cytokines on alternate days. After 7 days, the immature dendritic cells resulting from this procedure express a phenotype CD83⁻, CD14^{low} or CD14⁻, HLA-DR⁺, as was demonstrated by FACS analysis.

Immature DCs were matured by culturing the cells in a medium containing 100 ng/ml TNF-a for 3 days, after which, they expressed CD83 on their cell surface.

### Example II

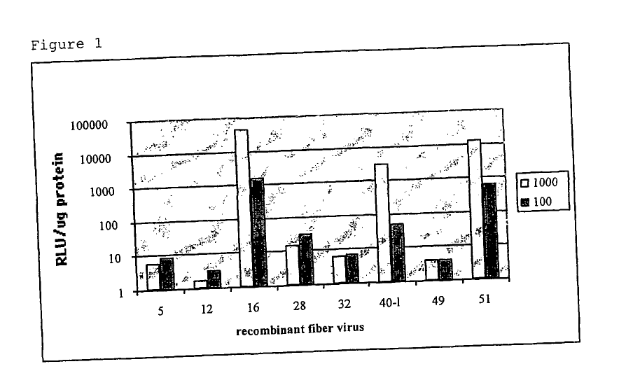

5×10⁵ immature DCs were seeded in wells of 24-well plates and exposed for 24 hours to 100 and 1000 virus particles per cell of each fiber recombinant virus. Virus tested was Ad5, and the fiber chimeric viruses based on Ad5: Ad5.Fib 12, Ad5.Fib16, Ad5.Fib28, Ad5.Fib32, Ad5.Fib40-L (long fiber of serotype 40), Ad5.Fib49, and Ad5.Fib51 (where Fibxx stands for the serotype from which the fiber molecule is derived). These viruses are derived from subgroup C, A, B, D, D, F, D, and B respectively. After 24-hours, cells were lysed (1% Triton X-100/PBS) and luciferase activity was determined using a protocol supplied by the manufacturer (Promega, Madison, Wis. USA). The results of this example, shown in FIG. 1, demonstrate that Ad5 poorly infects immature DCs as evidenced by the low level of transgene expression. In contrast, Ad5.Fib16 and Ad5.Fib51 (both a B-group fiber chimeric virus) and also Ad5.Fib40-L (Subgroup F) show efficient infection of immature DCs based on luciferase transgene expression.

### Example III

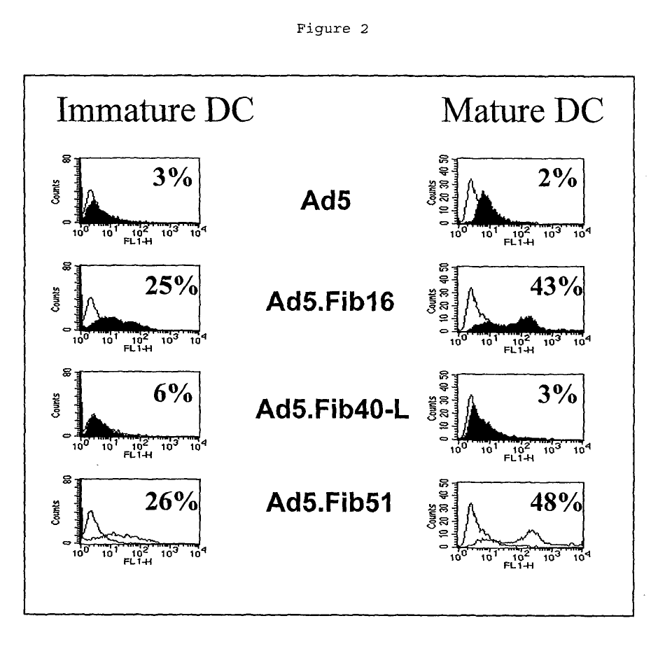

In a second experiment, 5×10⁵ immature and mature dendritic cells were infected with 10,000 virus particles per cell of Ad5, Ad5.Fib16, Ad5.Fib40-L, and Ad5.Fib51 all carrying the LacZ gene as a marker. LacZ expression was monitored by flow cytometric analysis using a CM-FDG kit system and the instructions supplied by the manufacturer (Molecular Probes, Leiden, NL). The results of this experiment, shown in FIG. 2, correlate with the previous experiment in that Ad5.Fib16 and Ad5.Fib51 are superior to Ad5 in transducing mature and immature human DCs. Also, this example shows that Ad5.Fib40-L is not as good as Ad5.Fib16 and Ad5.Fib51, but is better than Ad5.

### Example IV

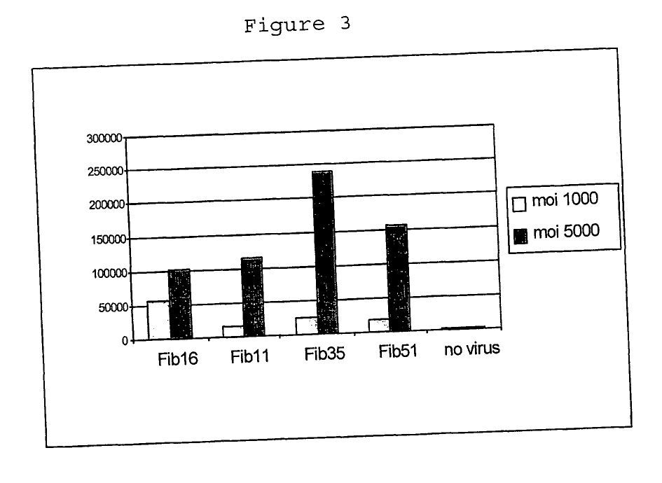

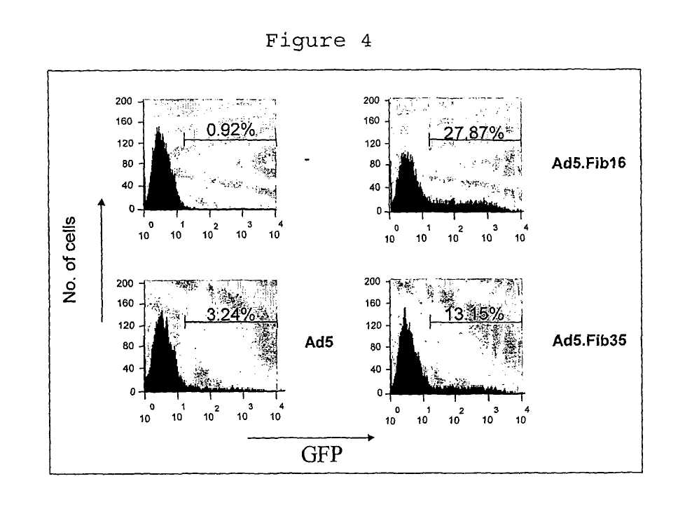

Based on the earlier Examples, we tested other chimeric adenoviruses containing fibers of B group viruses, for example, Ad5.Fib11 and Ad5.Fib35 for their capacity to infect DCs. We focused on immature DCs, since these are the cells that process an expressed transgene product into MHC class I and II presentable peptides. Immature DC's were seeded at a cell density of 5×10⁵ cells/well in 24 well plates (Costar) and infected with 1,000 and 5,000 virus particles per cell after which the cells were cultured for 48 hours under conditions for immature DCs prior to cell lysis and Luciferase activity measurements. The results of this Example, shown in FIG. 3, demonstrate that Ad5 based chimeric adenoviruses containing fibers of group-B viruses efficiently infect immature DCs. In a fourth experiment, we again infected immature DCs identically as described in the former experiments but this time Ad5, Ad5.Fib16, and Ad5.Fib35 were used carrying GFP as a marker gene. The results on GFP expression measured with a flow cytometer 48 hours after virus exposure are shown in FIG. 4, and correlate with the data obtained so far. Thus, the results so far are consistent in that Ad5 based vectors carrying a fiber from a alternative adenovirus derived from subgroup B predominantly fiber of 35, 51, 16, and 11 are superior to Ad5 for transducing human DCs.

### Example V

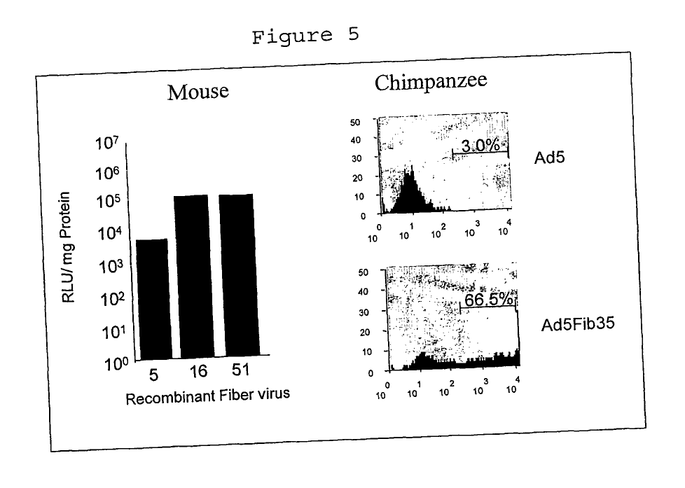

The adenoviruses disclosed herein are also very suitable for vaccinating animals. To illustrate this, we tested DCs derived from mice and chimpanzees to identify whether these viruses could be used in these animal models. Chimpanzees in particular, since the receptor for human adenovirus derived from subgroup B is unknown to date and therefore it is unknown whether this protein is conserved among species. For both species, immature DCs were seeded at a density of 10⁵ cells per well of 24-well plates. Cells were subsequently exposed for 48 hours to 1000 virus particles per cell of Ad5, Ad5Fib16, and Ad5.Fib51 in case of mouse dendritic cells and Ad5, and Ad.Fib35 in case of chimpanzee DCs (see, FIG. 5). The mouse experiment was performed with viruses carrying luciferase as a marker, and demonstrated approximately 10-50 fold increased luciferase activity as compared to Ad5.

The chimpanzee DCs were infected with the GFP viruses, and were analytes using a flow cytometer. These results (also shown in FIG. 5) demonstrate that Ad5 (3%) transduces chimpanzee DCs very poorly in comparison to Ad5.Fib35 (66.5%).

### Example VI

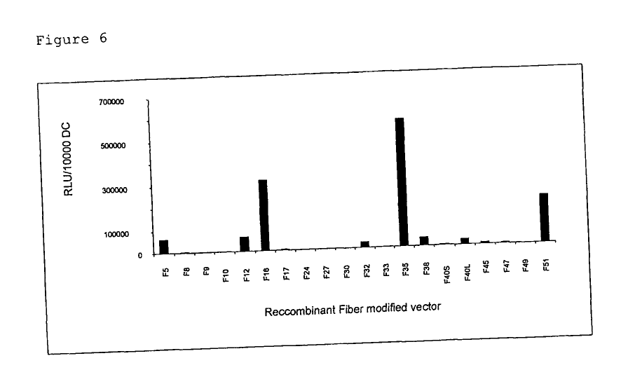

Immature DCs were incubated with Ad5.Luc or with the fiber-modified vectors at a virus dose of 10⁵virus particles per DC. Luciferase transgene expression was determined 24 hours after virus exposure. Results are a representative of two independent experiments performed with DC derived from 2 different individuals, and depicted graphically in FIG. 6. Results are expressed in relative light units (“RLU”) per 10⁴ DC versus recombinant fiber modified vector.

### Example VII

Following-up on Example V, immature DCs were incubated with Ad5.GFP or Ad5Fib16.GFP, Ad5Fib35,GFP, Ad5fib40-L.GFP, or Ad4.Fib51.GFP. Different dosages were used: 10³, 10⁴, or 10⁵ virus particles per DC (white bar, grey bar, or black bar respectively in FIG. 7). GFP expression was determined 24 hours after virus exposure. FIG. 7 expresses the results in Graph a as percentage GFP positive cells and, in Graph b, as median fluorescence intensity. Results shown are derived from 3 independent experiments performed with DC derived from 3 different individuals.

### Example VIII

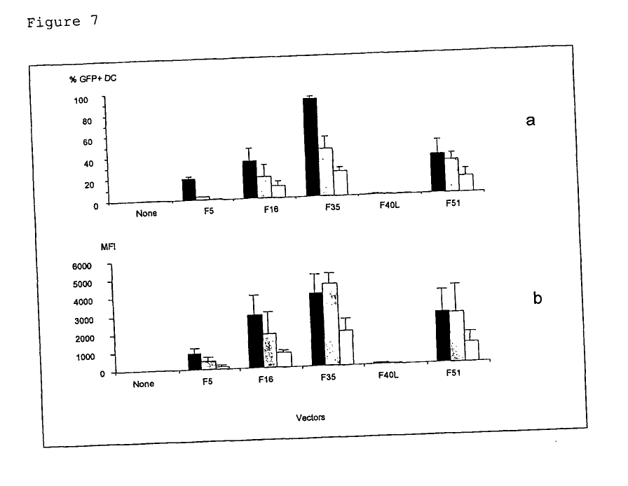

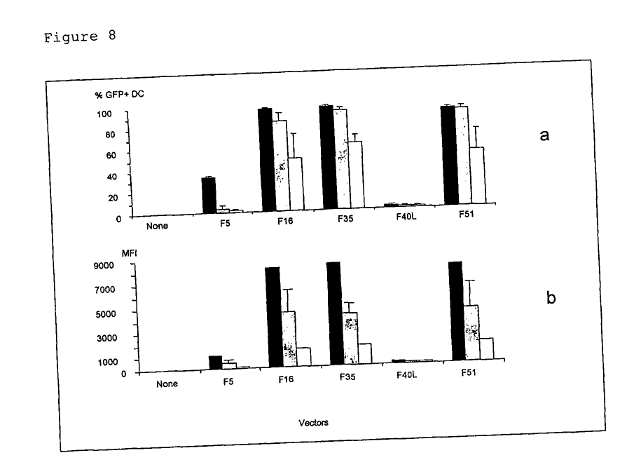

Immature DC were treated for 48 hours with LPS to allow maturation of the DC. Matured DCs were incubated with Ad5. GFP or Ad5Fib 16. GFP, Ad5Fib35. GFP, Ad5fib40-L.GFP, or Ad4.Fib51.GFP. Dosages used were 10³, 10⁴, or 10⁵ virus particles per DC (depicted as white, grey, and black bars respectively in FIG. 8). GFP expression was determined 24 hours after virus exposure. In FIG. 8, results are expressed as (a) percentage GFP positive cells and (b) median fluorescence intensity (b). Results shown are derived from 3 independent experiments performed with DC derived from 3 different individuals.

### Example IX

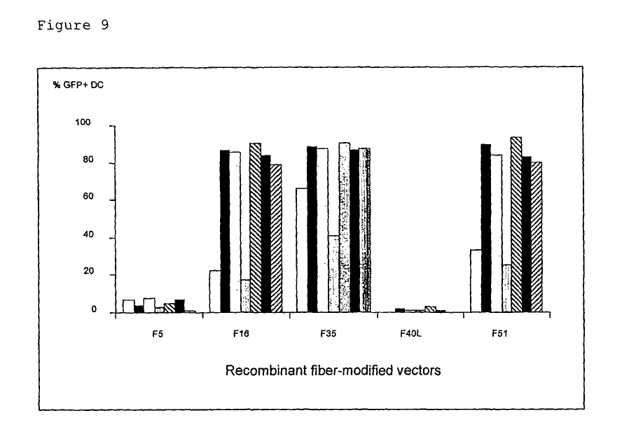

Immature dendritic cells were exposed for 48 hours to different maturation agents before being exposed to various viruses. The virus dosage used was 10⁴ virus particles per DC. GFP expression was determined 24 hours after virus exposure. The results are expressed as percentage GFP positive cells. Results shown are representative of 2 independent experiments. The results are graphically depicted in FIG. 9. The maturation agents used were LPS (black bars), TNF-a (white with black dots), MCM (diagonal downward), poly I:C (black with white dots), and anti-CD40 antibodies (diagonal upwards). As a negative control for a maturation marker, IFN-a (grey bars) was used, while immature dendritic cells were used as a general control (white bars).

### Example X

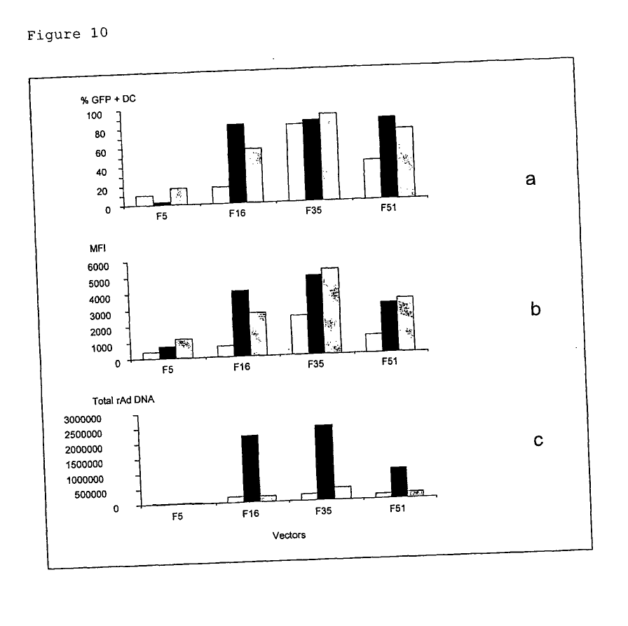

Dendritic cells were exposed to 10⁴ virus particles per cell where various vectors (F5, F16, F35, and F51) were used. The results are graphically depicted in FIG. 10, wherein, in (a), the percentage GFP positive cells detected is depicted, in (b) the median fluorescence intensity is depicted and, in (c), cells that were frozen and genomic DNA extracted to quantify the number of adenoviral genomes using real-time PCR. The DC types were immature DC (white bar), mature DC (black bar), or immature DC transduced and subsequently matured using LPS. Cells were analyzed for GFP expression 48 hours after virus exposure. The data are representative for two independent experiments.

### Example XI

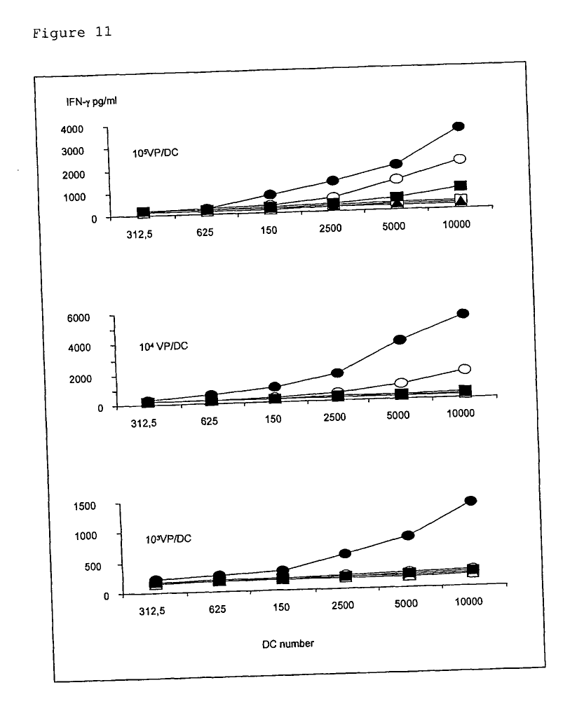

As graphically depicted in FIG. 11, immature dendritic cells were transduced with 10³, 10⁴ or 10³ virus particles (top, middle, and bottom graphs respectively) of Ad5.gp100 or Ad5.Fib35.gp100 (white squares and circles, respectively). Likewise, matured DC were transduced with 10⁵, 10⁴ or 10³ virus particles of Ad5.gp100 or Ad5.Fib35.gp100 (black squares and circles respectively). Transduced DC (10⁴ cells) were cultured with the HLA-A2-restricted 8J CTL clone (10⁴ cells) and IFN-gamma production which demonstrates CTL activation, which was determined 24 hours later.

### Example XII

Immature DC and mature DC (LPS) were exposed to 10⁴ virus particles per dendritic cell. Forty-eight hours after virus exposure, the cells were analyzed for expression of membrane proteins CD86, CD83, HLA-class I, and HLA-DR. Also, release of IL-12 was measured. As a control, non-transduced immature DC and mature DC were used. Results for the membrane proteins are expressed in mean fluorescence intensity (Table 1). Results for IL-12 production are expressed in pg/ml.

### Example XIII

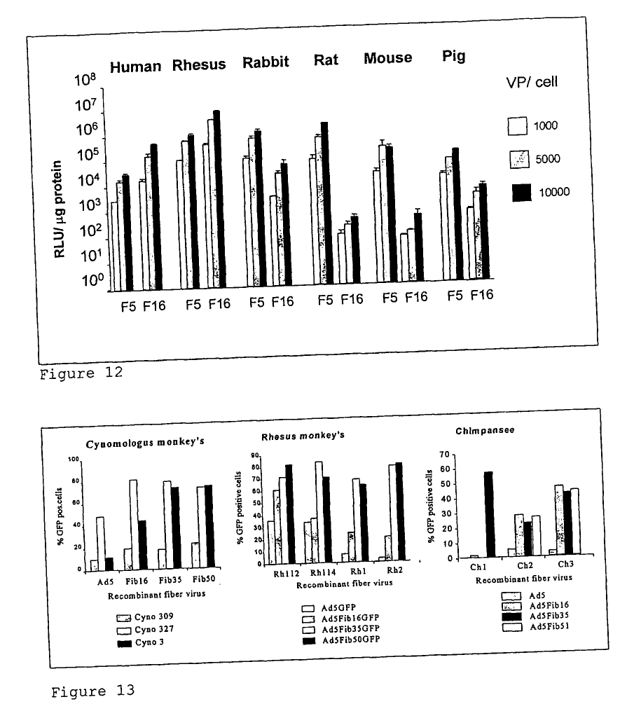

Receptor conservation in species: Testing of dendritic cells derived from human, chimpanzee, or mouse demonstrated, as described earlier, that an intrinsic difference exists between mice and chimpanzees in terms of expression patterns or conservation of human B-group adenovirus cellular attachment molecules. Earlier data demonstrated that Ad5Fib 16 and Ad5Fib35 infect mouse dendritic cells less efficient as compared to Ad5, as observed by levels of luciferase activity obtained (see FIG. 5). In contrast, both in human dendritic cells and chimpanzee dendritic cells data indicated that Ad5Fib 16 and Ad5Fib35 are superior to Ad5 (FIGS. 4 and 5). This latter phenomenon is not only true for dendritic cells derived from mice because a similar observation was made using another cell type, i.e. smooth muscle cells cultured from the carotid artery of mice, rats, rabbits and pigs (FIG. 12). These data confirm that the attachment molecules utilized by human 13-group viruses are not conserved between species. To further investigate whether at least between humans and all non-human primates the receptor for human B-group viruses are conserved, dendritic cells were isolated from heparinized blood obtained from chimpanzee (n=3), cynomolgus monkeys (n=3), and rhesus monkeys (n=4). Hereto, peripheral blood mononuclear cells (PBMCs) were generated after ficoll treatment (Leucosept, Greiner) and CD14 positive cells, i.e. monocytes were obtained via magnetic bead sorting (Variomacs, Milteny, German) and the instructions provided by the manufacturer. Monocytes were subsequently cultured for 7 days in the presence of hGM-CSF (Novartis, 100 Units/ml) and IL-4 (Busywork, 100 ng/ml). Based on cellular morphology a good population of dendritic cells should be cultured using this protocol that is exactly the same as for human dendritic cell isolation. Dendritic cells were counted and seeded at a concentration of 10⁴ cells per well of 48-well plates. Cells were cultured for 48 hours after which cells were exposed to a virus concentration of 1000 virus particles per cell of Ad5, Ad5Fib16, Ad5Fib35, or Ad5Fib50, all vectors carrying GFP as a marker gene. Forty-eight hours after virus addition, cells were washed with PBS/1% BSA, harvested and analytes for GFP expression on a flow cytometer (Facscalibur, Becton Dickinson). Based on the percentage of cells positive for GFP, Ad5Fib 16, Ad5Fib35, and Ad5Fib50 in all samples tested proved superior to Ad5 for the genetic modification of non-human primate dendritic cells (FIG. 13 panels A to C). Thus a thorough investigation among different species identified an intrinsic difference between rodents and pigs on one hand, and humans and non-human primates on the other hand. Moreover, it could be concluded that determination of vector superiority of Ad5Fib 16, Ad5Fib35, and Ad5Fib50 over Ad5 can only be determined using non-human primate models.

### Example XIV

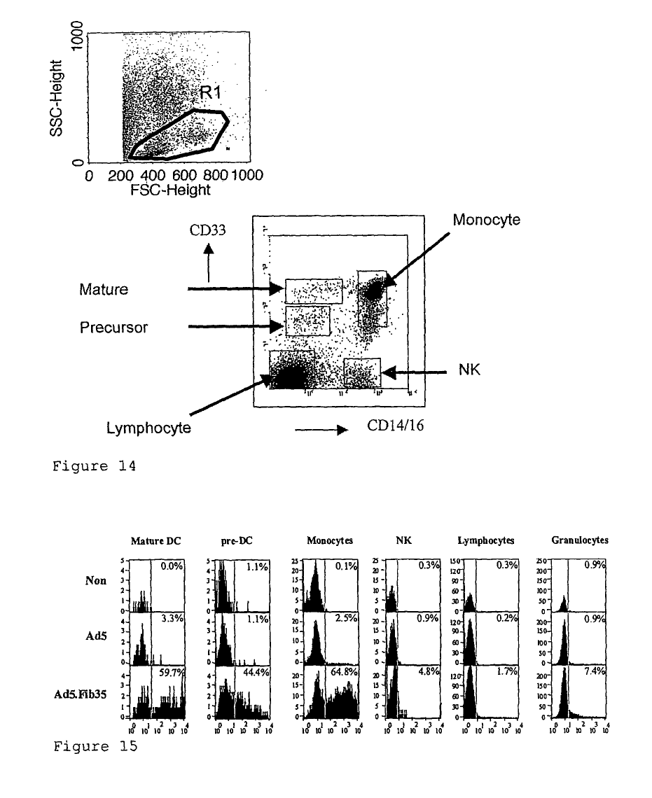

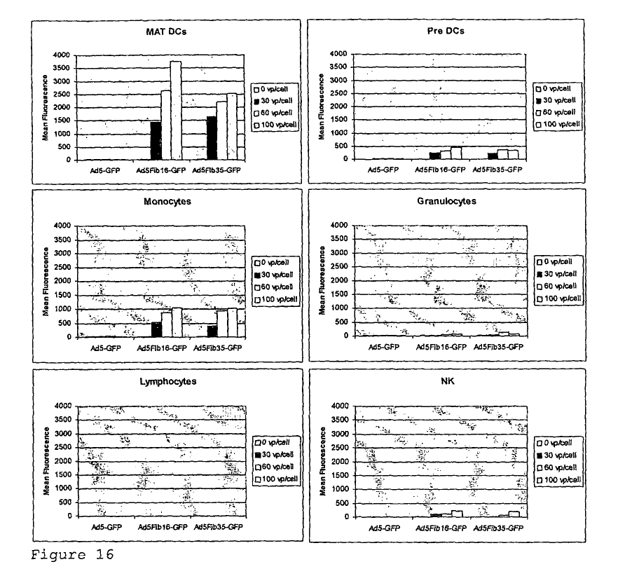

Vector specificity for dendritic cells residing in human blood. So far, data has shown that improved vectors as compared to Ad5, i.e. Ad5Fib15, Ad5Fib35, and Ad5Fib50 were identified. Improvements were found in the ability of the fiber-chimeric viruses to infect human monocyte-derived dendritic cells resulting in an improved T-cell activation when transferring model antigens such as the melanoma antigen gp100 (see FIG. 11). For direct in vivo use of adenoviral vectors, ideally only monocytes and mature/immature dendritic cells residing in the blood should be infected. To investigate the specificity of one the improved vectors identified, we set-up a panel of experiments utilizing human PBMCs. In the first experiment different sub-populations of cells present in the complex cell population of PBMCs were investigated. Hereto, 30 ml of human blood (derived from a buffycoat) was depleted of erythrocytes using erythrocyte lysis buffer (1 mM EDTA, 1.7 mM NH4Cl, pH 7.3). PBMCs were subsequently seeded at a concentration of 106 cells per well of 24-well plates and exposed to Ad5-GFP, Ad5Fib16-GFP or Ad5Fib35-GFP using virus dosages of 100, 1000 or 5000 virus-particles (vp) per cell. Infection was allowed for 1,5 hour at 37° C. in a 10% CO₂-incubator, cells were washed with medium to remove remaining viruses and were subsequently cultured overnight in RPMI/10% FBS/pen-strep allowing expression of the GFP-construct. The next day, cells were stained with CD33-PerCP/Cy5 and CD14/CD16-PE, to visualize the individual sub-populations present in human blood, when gated on the monocytes and lymphocytes in the FSC/SSC plot (monocytes, mature DCs, precursor DCs, natural killer cells (NK) and lymphocytes (Thomas et al 1993, Thomas and Lipsky, 1994). A general picture showing the flow cytometric separation of different cell populations is shown in FIG. 14. Cells expressing the GFP-construct were visualized in the FL1-channel using the FACS-Calibur flow cytometer. Mature DCs, precursor DCs and monocytes were efficiently transduced with Ad5Fib35-GFP using a very low vector dose (100 vp/cell). At this virus concentration, no GFP positive cells could be detected after genetic modification with Ad5-GFP (see FIG. 15). Because of the enormous efficient infection obtained with Ad5Fib35 this experiment was repeated using even lower dosages of vector to find the lower limit in the ability of Ad5Fib35 to infect dendritic cells residing in the blood. Moreover, Ad5Fib16 was taken along to identify the potency of this vector as compared to Ad5 and Ad5Fib35. The results from this experiment, expressed in mean GFP fluorescence obtained, clearly demonstrates that both Ad5Fib16 and Ad5Fib35 are superior as compared to Ad5 to infect monocytes, pre-dendritic cells and mature dendritic cells residing in the blood. Moreover, Levels of GFP transgene expression are high and readily detectable by flow cytometry even at vector dosages as low as 30 virus particles per cell, indicative for extremely efficient uptake of Ad5Fib16 and Ad5Fib35 by these cell types.

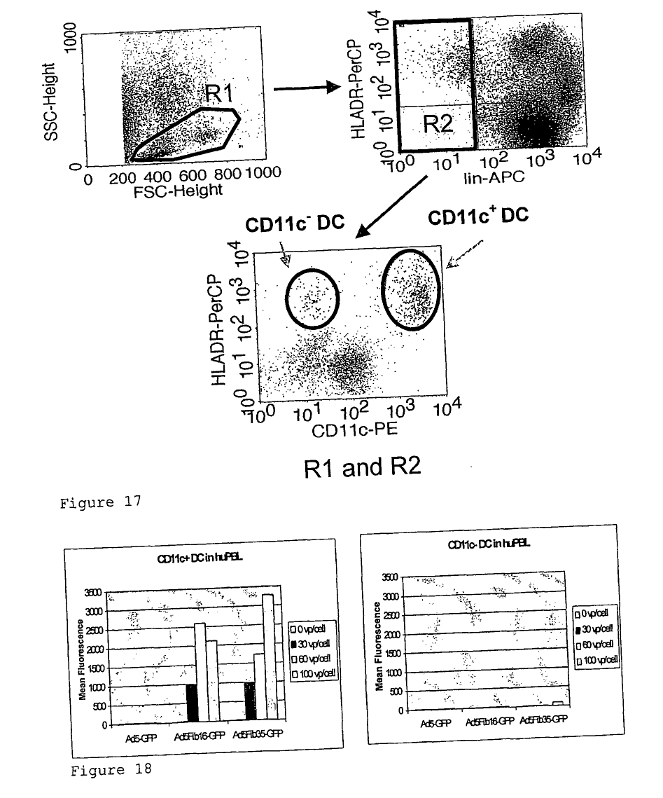

To further dissect in PBMCs the specificity of Ad5Fib16 and Ad5Fib35 a next set of experiments was conducted. These experiments were based on the knowledge that two types of dendritic cells reside in human blood (see, Table 2). The two types of dendritic cells are distinguished on the basis of morphology, phenotype, antigen handling capacity and most importantly, they may represent two distinct lineages of antigen presenting cells (O'Doherty et al 1994; Robinson et al, 1999). To determine which DC type (the myeloid or the lymphoid DCs), in human blood are infected with the different recombinant viruses the following experiments were performed. Human blood (derived from a buffycoat) was depleted for erythrocytes as described above to and PBMCs were seeded at a concentration of 10⁶ cells per well of 24-well plates. Twenty-four hours later cells were exposed to Ad5-GFP, Ad5Fib16-GFP and Ad5Fib35-GFP using a vector dose of 0, 30, 60 or 100 vp/cell. Infection was allowed for 2 hours, cells were washed to remove residual viruses, and cells were subsequently cultured overnight in RPMI/10% FBS/pen-strep at 37° C. in a 10% CO₂-incubator, allowing expression of the GFP-construct. The next day, cells were stained with all lineage markers (CD3-APC, CD14-APC, CD16-APC, CD19-APC, CD56-APC), HLADR-PERCP and CD11c-PE to visualize the CD11c⁺ (myeloid) and CD11c⁻ (lymphoid) DC in blood, when gated on the monocytes and lymphocytes in the FSC/SSC plot). A general overview of the flow cytometric settings required to visualize both dendritic cell populations present in PBMCs is shown in FIG. 17. Next, cells expressing the GFP-construct were visualized in the FL1-channel using the FACS-Calibur flow cytometer. Baaed on the results obtained two clear conclusions could be drawn: a) Ad5Fib35 and Ad5Fib16 again proof much more potent as compared to Ad5 to infect dendritic cells derived from the human blood, and b) There is a clear preference of Ad5Fib16 and Ad5Fib35 for dendritic cells of myeloid origin.

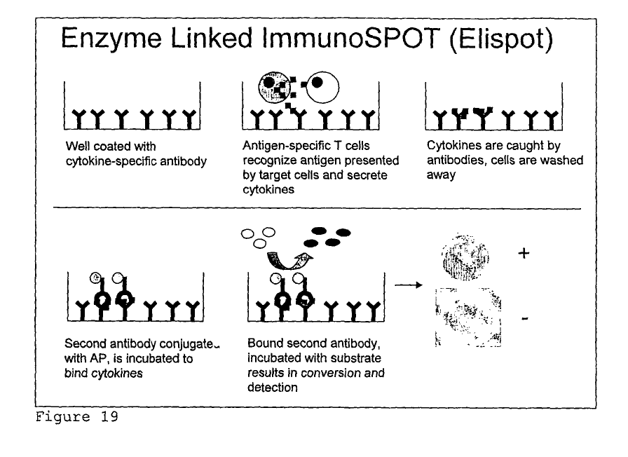

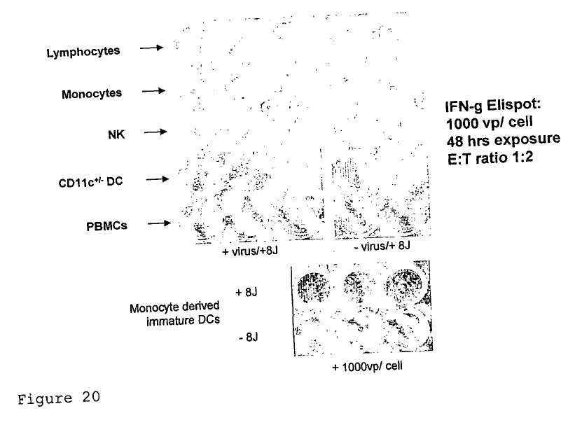

For final proof of concept, human blood sub populations of cells were isolated using a Facs sorter (FacsVantage, Becton Dickinson) and sorted cells were seeded in wells of 96-well plates at a concentration of 10⁴ cells per well. Sorted and seeded cells were subsequently exposed to 1000 virus particles per cell of Ad5Fib35 carrying the model antigen gp100, a tumor-antigen for which a specific T-cell clone is available (clone 8J, see also FIG. 11). Cells were subsequently incubated in RPMI/10% FBS/pen-strep/gentamycin (60 ug/ml)/GM-CSF (Novartis, 800 IU/ml)/IL-3 (PreproTech, 100 U/ml) for 48 hours at 37° C. in a 10% CO2 incubator, allowing expression and processing of the gp100 epitope. GM-CSF and IL-3 were added to the medium to allow longer survival of the sorted DCs (Strobl et al 1998), whereas gentamycin was added to prevent eventual infections due to the semi-sterile sorting procedure. Positive controls were taken along, using cultured monocyte derived DCs that were infected with Ad5Fib35-gp100 as has been described earlier (see FIG. 11). After 48 hours, residual virus was removed and the gp100-specific CTL (clone 8J) was added in at a cell density of 5000 cells/well in a multiscreen 96-wells plate (Millipore) pre-coated overnight at 4° C. with rat-anti-mouse Interferon-gamma (effector to target cell ratio is thus 1:2). As negative controls non-infected sorted cells were taken. The production of interferon-gamma by the gp100 specific T-cells was allowed to proceed for 24 hours at 37° C. in a 10% CO₂ incubator. After 24 hours, the T-cells of 8J activated by gp100 expressing sorted cells and thus producing interferon-gamma were detected using a Elispot kit specific for interferon-gamma. The principle of the Elispot assay and the materials used is given in FIG. 19. From the results obtained (FIG. 20) on the distinct sorted cell populations several conclusions can be drawn. A) Selection of possible improved vectors from the fiber-chimeric vector library using viruses carrying marker genes such as GFP, LacZ, and Luciferase is a valid strategy since selected vectors indeed are potent in eliciting biological responses. B) Infection results obtained with GFP correlate with biological activity since interferon gamma producing and thus activating T-cells are only found in sorted monocytes and dendritic cell populations. C) These results demonstrate and validate the capacity of dendritic cells, selected on their flow cytometric phenotype, to trigger immune responses thus clearly showing that these cells are true antigen presenting cells, i.e. dendritic cells. All in all, the results generated both on monocyte derived dendritic cells and blood derived dendritic cells thus correlate and identifies Ad5Fib 16, Ad5Fib35, and Ad5Fib50 as potent vectors that can be used a vaccine delivery vehicles. Hereto, as described earlier, these adenoviral vectors can be engineered to deliver and express antigenic proteins to antigen presenting cells in the body that trigger a potent cellular and humoral immune response against the antigenic proteins transferred. Antigenic proteins of interest to battle human diseases can be derived from viruses (HIV, HPV, Ebola), parasites (malaria) or can be proteins identified as proteins only expressed on certain human tumor cells. Thus the potential vaccine fields in which the improved adenoviral vectors can play a role as an important tool to prevent or treat many human diseases is considered high.

TABLE 1
Effect of fiber-modified adenoviral vectors on the ability of DC to
maturate.

| | | | | | IL-12 p70 |
|---|---|---|---|---|---|
| DC type | CD86* | HLA-DR | HLA-class I* | CD83* | (pg/ml) |
| IDC | 99 | 724 | 553 | 8 | 0 |
| LPS | 1786 | 1697 | 1906 | 96 | 324 |
| F5 | 232 | 889 | 627 | 17 | 0 |
| F5 LPS | 2020 | 2392 | 2439 | 107 | 3647 |
| F16 | 460 | 1038 | 769 | 19 | 0 |
| F16 LPS | 2474 | 2152 | 2694 | 143 | 5628 |
| F35 | 751 | 1278 | 750 | 34 | 0 |
| F35 LPS | 2298 | 2380 | 2360 | 157 | 12944 |
| F51 | 446 | 1086 | 638 | 19 | 0 |
| F51 LPS | 2633 | 2502 | 2351 | 132 | 9267 |

TABLE 2
Differences between CD11c⁺ and CD11c⁻ DC's in human blood.
* Monocyte-derived DC = CD11C⁺ DC = DC1 = Mo-DC = myeloid DC

CD11C = β2-integrin
Markers: CD3⁻, CD14⁻, CD16⁻, CD19⁻, CD34⁻, il-3-R = CD123^{low},
CD1a⁻, CD83^{-,} CD10⁻, CD45RO⁺, CD13⁺, CD33⁺, GM-CSF-R⁺,
MHC class II⁺⁺⁺, CD2⁺, CD32⁺, CD62L⁺, myeloid markers⁺
Origin: derived from tissues, already activated ® may be en route to the
spleen or lymph nodes
Function/phenotype:
Able to take up antigen (25-54% of CD11c⁺ cells) which is enhanced after
culturing.
When Mo-DC are incubated with T cells ® T cells will produce IFN-γ
Mo-DC can develop after culturing with GM-CSF/TNFα into: CD11c⁺,
CD13⁺, CD33^{+/-}, CD4⁻, CD1a⁺, CD83⁺, CD9⁺
Mo-DC have a potent T cell stimulatory activity (MLR)
Irregularly shaped, hyperlobulated nucleus
May give rise to DC and macrophages found in peripheral tissue

* Plasmacytoid DCs = CD11C⁻ DC = DC2 = P-DC = lymphoid DC

Markers: MHC-class II⁺⁺, CD4⁺, CD10⁺, il-3-R⁺ = CD123⁺⁺,
CD45RA⁺, CD10^{+/-}, CD62L⁺⁺ (selectin responsible for homing of naive
lymphocytes to lymph nodes) CD3⁻, CD14⁻, CD16⁻, CD19⁻, CD45RO⁻,
CD34⁻, CD1a⁻, CD83⁻
Origin: marrow-derived pre DC underway to the tissues immature
Function/phenotype:
immature state, require Mo-derived cytokines for DC maturation
lymphoid morphology: Rounded with oval or indented nucleus, prominent
perinuclear pale zone
lack myeloid markers
IFN-a producers
give rise to plasmacytoid T cells of secondary lymphoid tissue
P-DC + T cells: IL-4 production by T cells
P-DC require stimulation in lymph nodes via CD40L provided by T cells,
that are earlier activated by antigen-presenting mo-DCs ® further
maturation of P-DC, secretion of IL-12, maintain production of IFN-α
−> may prolonge Th1-phase
Can develop after culturing with GM-CSF/TNFa into CD11C⁻, CD13⁻,
CD33⁻, CD4⁺, CD1a⁻, CD83^{+/-}

Potent in MLR, but less as with CD11c⁺ DC

Culture with M-CSF->apoptosis

Although the invention has been described using a certain amount of detail, and through the use of preferred embodiments, the scope of the invention is to be determined by the appended claims.

### REFERENCES

Amin et al. “Nitric Oxide Synthase and Cyclooxygenase: Distribution, Regulation and Intervention in Arthritis” Curr Opin Rheumatol. 11, pp. 202-209 (1999).

Arnberg et al. “Fiber Genes of Adenoviruses with Tropism for the Eye and the Genital Tract”, Virology 227:239-244 (1997).

Bergelson, et al. “Isolation of a Common Receptor for Coxsackie B Virus and Adenoviruses 2 and 5”, Science 275:1320-1323 (1997).

A. Bout “Gene Therapy”, pp. 167-182, in: D. J. A. Crommelin and R. D. Sindelar (editors), Pharmaceutical Biotechnology, Harwood Academic Publishers (1997).

A. Bout “Prospects for Human Gene Therapy” Eur. J. Drug Met. and Pharma, 2:175-179 (1996).

Blaese et al. Cancer Gene Ther. 2:291-297 (1995).

Brody, S. L. and Crystal, R. G. “Adenovirus Mediated in Vivo Gene Transfer” Ann. N. Y. Acad Sci. 716:90-101 (1994).

Chroboczek et al. “Adenovirus fiber”, pp. 163-200 in: W. Doerfler and P. Bohm (editors), The Molecular Repertoire of Adenoviruses, I. (Springer-Verlag, Berlin, 1995).

Defer et al. “Human Adenovirus-host Cell Interactions: Comparative Study with Members of Subgroup B and C” Journal of Virology, 64 (8):3661-3673 (1990).

Fallaux et al. “New Helper Cells and Matched E1-deleted Adenovirus Vectors Prevent Generation of Replication Competent Adenoviruses” Human Gene Therapy, 9, pp.1909-1917 (1998).

Fernandez et al. “In Vivo Transfer of Interleukin-1 Receptor Antagonist Gene in Osteoarthritic Rabbit Knee Joints: Prevention of Osteoarthritis Progression”, Am J Pathol, 154, pp.1159-1169 (1999).

Francki et al. “Classification and Nomenclature of Viruses. Fifth Report of the International Committee on Taxonomy of Viruses”, Arch. Virol. Suppl., 2:140-144 (1991).

Frenkel, S R and DiCesare, P E “Degradation and Repair of Articular Cartilage”, Front Biosci 15(4):671-685 (1999).

Gazit et al. “Engineered Pluripotent Mesenchymal Cells Integrate and Differentiate in Regenerating Bone: a Novel Cell-mediated Gene Therapy”, J Gene Med I, pp.121-133 (1999).

Gall et al. “Adenovirus Type 5 and 7 Capsid Chimera: Fiber Replacement Alters Receptor Tropism Without Affecting Primary Immune Neutralization Epitopes”, Journal of Virology 70(4):2116-23 (1996).

Goto et al. “Transfer of LacZ marker gene to the meniscus”, J Bone Joint Surg Am, 81:918-925 (1999).

Greber et al. “Stepwise Dismantling of Adenovirus 2During Entry into Cells”, Cell 75:477-486 (1993).

R. O. Hynes “Integrins: Versatility, Modulation and Signaling in Cell Adhesion”, Cell 69:11-25 (1992).

Herz, J. and Gerard, R. D. “Adenovirus-mediated Transfer of Low Density Lipoprotein Receptor Gene Acutely Accelerates Cholesterol Clearance in Normal Mice”, Proc. Natl. Acad. Sci. U.S.A., 96 2812-16 (1993).

J. C. Hierholzer, “Adenovirus in the Immunocompromised Host”, Clin. Microbiol Rev., 5:262-74 (1992).

Hierholzer et al. “Adenoviruses from Patients with AIDS: A Plethora of Serotypes and a Description of Five New Serotypes of Subgenus D (Types 43-47)”, J. Infect. Dis., 158:804-813 (1988).

Hong et al. “Adenovirus Type 5 Fiber Knob Binds to MHC Class 1 (2 Domain at the Surface of Human Epithelial and B Lymphoblastoid Cells”, EMBO J. 16:2294-2306(1997).

Hsu et al. “A Monoclonal Antibody Specific for the Cellular Receptor for the Group B Coxsackieviruses”, J. Virol 62(5): 1647-52 (1988).

Huard et al. “The Route of Administration Is a Major Determinant of the Transduction Efficiency of Rat Tissues by Adenoviral Recombinants” Gene Ther. 2: 107-115 (1995).

Ishibashi, M. and Yasue, H. “The Adenoviruses,” H. S. Ginsberg, ed., Chapter 12,497-561 (Plenum Press, London, New York. 1984).

Kass-Eisler et al. “The Impact of Developmental Stage, Route of Administration and the Immune System on Adenovirus-mediated Gene Transfer”, Gene Ther., 1:395402 (1994).

Khoo et al. “Adenovirus Infections in Human Immunodeficiency Virus-positive Patients: Clinical Features and Molecular Epidemiology”, J. Infect. Dis 172, 629-637 (1995).

Kidd et al. “Adenovirus Type 40 Virions Contain Two Distinct Fibers”, Virology, 192:73-84 (1993).

Kramer et al, “Embryonic Stem Cell-derived Chondrogenic Differentiation in V Activation by BMP-2 and BMP-4” Mech Dev 92:193-205.

Krasnykh et al. “Generation of Recombinant Adenovirus Vectors with Modified Fibers for Altering Viral Tropism”, J. Virol, 70(10):6839-6846 (1996).

Krasnykh et al. “Characterization of an Adenovirus Vector Containing a Heterologous Peptide Epitope in the Hi Loop of the Fiber Knob” J. Virol. 72(3): 1844-1852 (1998).

Law et al. “Infection of Primary CNS Cells by Different Adenoviral Serotypes: Searching for a More Efficient Vector”, Abstract 1^{st} Annual Meeting American Society of Gene Therapy, Seattle, Wash. (1998).

K. N. Leppard “E4 Gene Function in Adenovirus, Adenovirus Vector and Adeno-associated Virus Infections”, J. Gen. Virol., 78:2131-2138 (1997).

Lubberts et al. “Adenoviral Vector-mediated Gene Delivery to the Temporomandibular Joint in Guinea-pigs”, Arch. Oral Biol, 44:701-709 (1999).

Madry, H and Trippel, S B “Efficient Lipid-mediated Gene Transfer to Articular Chondrocytes” Gene Ther, 7:286-291 (2000).

Mason et al. “Expression of Human Bone Morphogenesis Protein 7 in Primary Rabbit Periosteal Cells: Potential Utility in Gene Therapy for Osteochondral Repair”, Gene Ther 5:1098-1104 (199_).

Morgan et al. “Structure and Development of Viruses as Observed in the Electron Microscope.x. Entry and Uncoating of Adenovirus”, J. Virol 4, 777-796 (1969).

Nishida et al. “Modulation of the Biological Activity of the Rabbit Intervertebral Disc by Gene Therapy: an in Vivo Study of Adenovirus-mediated Transfer of the Human Transforming Growth Factor Beta 1 Encoding Gene”, Spine 24:2419-2425 (1999).

O'Doherty U, Peng M, Gezelter S, Swiggard W J, Betjes M, Bbardwaj N, Steinman R M. Human blood contains two subsets of dendritic cells, one immunologically mature and the other immature. Immunology 82: 487-493, 1994.

Pizette, S and Niswander, L “BMPs are Required at Two Steps of Limb Chondrogenesis: Formation of Prechondrogenic Condensations and Their Differentiation into Chondrocytes”, Dev Biol 219:237-249 (2000).

Rea D, Havenga M J E, Van den Assem M, Sutmuller R P M, Lemckert A, Hoeben R C, Bout A, Melief C J M, Offringa R. Highly efficient transduction of human monocyte-derived dendritic cells with subgroup B fiber-modified adenovirus vectors enhances transgene-encoded antigen presentation to cytotoxic T cells. J Immunol 166: 5236-5244, 2001.

Richardson et al. “Repair of human cartilage after implantation of autologous chondrocytes”, J Bone Joint Surg. Br 81:1064-1068 (1999).

Robinson S P, Patterson S, English N, Davies D, Knight S C, Reid C D L. Human preipheral blood contains two distinct lineages of dendritic cells. Eur J Immunol 29: 2769-2778, 1999.

Roelvink et al. “Comparative Analysis of Adenovirus Fiber-cell Interaction: Adenovirus Type 2 (Ad2) and Ad9 Utilize the Same Cellular Fiber Receptor but Use Different Binding Strategies for Attachment”, J. Virol., 70:7614-7621 (1996).

Roelvink et al. “The Coxsackie-adenovirus Receptor Protein Can Function as a Cellular Attachment Protein for Adenovirus Serotypes from Subgroups A, C, D, E, and F”, J. Virol. 72:7909-7915 (1998).

Rogers et al. “Use of a Novel Cross-linking Method to Modify Adenovirus Tropism”, Gene Ther. 4:1387-1392 (1997).

Risau, W. “Mechanisms of Angiogenesis”, Nature, 386:671-674 (1997).

Schulick, A H., Vassalli, G., Dunn, P. F., Dong, G., Rade, J. J., Zarnarron, C. and Dichek, D. A. “Established Immunity Precludes Adenovirus-mediated Gene Transfer in Rat Carotid Arteries”, (1997).

Schnurr, D and Dondero, M. E. “Two new candidate adenovirus serotypes”, Intervirol., 36:79-83 (1993).

Shabram et al. Analytical Anion-Exchange HPLC of Recombinant Type-5 Adenoviral Particles”, Hum. Gene Ther., 8(4):453-465 (1997).

Silverman et al. “Adhesion of Tissue-engineered Cartilage to Native Cartilage”, Plast Reconstr. Surg 105:1393-1398 (2000).

Signas et al. “Adenovirus 3 Fiber Polypeptide Gene: Complications for the Structure of the Fiber Protein”, J. Virol. 53:672-678 (1985).

Stevenson et al. “Human Adenovirus Serotypes 3 and 5 Bind to Two Different Cellular Receptors via the Fiber Head Domain”, J. Virol. 69(5):2850-2857 (1995).

Stevenson et al. “Selective Targeting of Human Cells by a Chimaeric Adenovirus Vector Containing a Modified Fiber Protein”, J. Virology 71(6):4782-4790 (1997).

Stouten et al. “New Triple Helical Model for the Shaft of the Adenovirus Fiber”, J. Mol. Biol., 226:1073-1084 (1992).

Strobl H, Scheinecker C, Riedl E, Csmarits B, Bello-Fernandez C, Pickl W F, Majdic O, Knapp W. Identification of CD68⁺lin⁻ peripheral blood cells with dendritic precursor characteristics. J Immunol 161: 740-748, 1998.

Svensson, V. and Persson, R “Entry of Adenovirus 2 into Hela cells”, J. Virol. 51:687-694 (1984).

Taniguchi et al. “Induction of the p16INK4a Senescence Gene as a New Therapeutic Strategy for the Treatment of Rheumatoid Arthritis”, Nat Med 5:760-767 (1999).

Thomas R, Davis L S, Lipsky P. Isolation and characterization of human peripheral blood dendritic cells. J Immuno 150: 821-834, 1993.

Thomas R, Lipsky P E. Human peripheral blood dendritic cell subsets. Isolation and characterization of precursor and mature antigen-presenting cells. J Immun 153:4016-4028, 1994.

P. C. van der Vliet “Adenovirus DNA Replication” in: W. Doerfler and P. Böhm (editors), The Molecular Repertoire of Adenoviruses II. (Springer-Verlag, Berlin 1995).

Varga et al. “Infectious Entry Pathway of Adenovirus Type 2”, J. Virol, 65:6061-6070 (1991).

G. Wadell “Molecular Epidemiology of Human Adenoviruses”, Curr. Top. Microbiol. Immunol. 110:191-220 (1984).

Whalen et al. “Adenoviral Transfer of the Viral IL-10 Gene Periarticularly to Mouse Paws Suppresses Development of Collagen-induced Arthritis in Both Injected and Uninjected Paws”, J Immunol, 162:3625-3632 (1999).

Wickham et al. “Targeting of Adenovirus Penton Base to New Receptors Through Replacement of its RGD Motif with Other Receptor-specific Peptide Motifs”, Gene Therapy 2:750-756 (1995).

Wickham et al. “Integrins avb3 and avb5 Promote Adenovirus Internalization but Not Virus Attachment”, Cell 73, 309-319 (1993).

Wold et al. “E3 Transcription Unit of Adenovirus” in: W. Doerfler and P. Böhm (editors), The Molecular Repertoire of Adenoviruses I, (Springer-Verlag, Berlin 1995).

Zabner et al. “Type 17 Fiber Enhances Gene Transfer”, Abstract 1^{st} Annual Meeting American Society of Gene Therapy, Seattle, Wash. (1998).

1. Use of a recombinant adenoviral vector for the delivery of a heterologous nucleic acid to a dendritic cell, wherein said recombinant adenoviral vector is provided with a tropism for dendritic cells.

2. Use according to claim 1, wherein said recombinant adenoviral vector has an at least partially reduced tropism for liver cells.

3. Use according to claim 1 or 2, wherein said tropism for dendritic cells is provided by at least a part of a virus capsid or a functional derivative and/or analogue thereof.

4. Use according to claim 3, wherein said virus capsid comprises proteins, or functional parts, derivatives and/or analogues thereof, from at least two different adenoviruses.

5. Use according to claim 4, wherein at least one of said adenoviruses is an adenovirus of subgroup B.

6. A dendritic cell provided with a heterologous nucleic acid through the use of a recombinant adenoviral vector according to any one of claims 1-5.

7. A method for transducing dendritic cells, comprising the steps of:
isolating dendritic cells from a donor;
culturing said dendritic cells; and
contacting said dendritic cells with a recombinant adenoviral vector comprising a heterologous nucleic acid;
wherein said recombinant adenoviral vector is provided with a tropism for dendritic cells.

8. A gene delivery vehicle having been provided with at least a tissue tropism for dendritic cells wherein said tissue tropism for dendritic cells is provided by a viral capsid protein.

9. The gene delivery vehicle of claim 8, wherein said tissue tropism is provided by viral capsid that comprises protein fragments derived from at least two different viruses.

10. The gene delivery vehicle of claim 9, wherein at least one of said at least two different viruses is an adenovirus.

11. The gene delivery vehicle of claim 10, wherein at least one of said at least two different viruses is an adenovirus of subgroup B.

12. The gene delivery vehicle of claim 9, wherein at least one of said protein fragments comprises a tissue tropism determining fragment of a fiber protein derived from a subgroup B adenovirus.

13. The gene delivery vehicle of claim 10, wherein at least one of said protein fragments comprises a tissue tropism determining fragment of a fiber protein derived from a subgroup B adenovirus.

14. The gene delivery vehicle of claim 11, wherein said subgroup B adenovirus is adenovirus 16.

15. The gene delivery vehicle of claim 12, wherein said subgroup B adenovirus is adenovirus 16.

16. The gene delivery vehicle of claim 13, wherein said subgroup B adenovirus is adenovirus 16.

17. The gene delivery vehicle of claim 12, further comprising protein fragments derived from an adenovirus of subgroup C.

18. The gene delivery vehicle of claim 13, further comprising protein fragments derived from an adenovirus of subgroup C.

19. The gene delivery vehicle of claim 14, further comprising protein fragments derived from an adenovirus of subgroup C.

20. The gene delivery vehicle of claim 8, comprising adenoviral nucleic acid, said adenoviral nucleic acid comprising at least one sequence encoding a fiber protein having at least a tissue tropism determining fragment of a subgroup B adenovirus fiber protein.

21. The gene delivery vehicle of claim 20, wherein said adenovirus nucleic acid is modified such that the capacity of said adenoviral nucleic acid to replicate in a target cell has been reduced or disabled.

22. The gene delivery vehicle of claim 20, wherein said adenoviral nucleic acid is modified such that the capacity of a host immune system to mount an immune response against adenovirus proteins encoded by said adenovirus nucleic acid has been reduced or disabled.

23. The gene delivery vehicle of claim 21, wherein said adenoviral nucleic acid is modified such that the capacity of a host immune system to mount an immune response against adenovirus proteins encoded by said adenovirus nucleic acid has been reduced or disabled.

24. The gene delivery vehicle of any one of claim 8, wherein said gene delivery vehicle comprises a minimal adenovirus vector or an Ad/AAV chimaeric vector.

25. The gene delivery vehicle of claim 8, further comprising at least one non-adenoviral nucleic acid.

26. The gene delivery vehicle of claim 10, wherein the adenovirus is Ad40L.

27. Use of a gene delivery vehicle according to any one of claims 8-26 for the delivery of a heterologous nucleic acid to a dendritic cell.

28. A method for transducing dendritic cells, comprising the steps of:
isolating dendritic cells from a donor;
culturing said dendritic cells; and
contacting said dendritic cells with a gene delivery vehicle according to any one of claims 8-26.

29. An adenovirus capsid having a tissue tropism for dendritic cells wherein said adenovirus capsid comprises:
proteins from at least two different adenoviruses; and
a tissue tropism determining fragment of a fiber protein derived from a subgroup B adenovirus.

30. A composition comprising a gene delivery vehicle having been provided with at least a tissue tropism for dendritic cells, said tissue tropism for dendritic cells being provided by a virus capsid, said virus capsid comprising protein fragments derived from at least two different viruses, wherein at least one of said at least two different viruses is an adenovirus of subgroup B.

31. The composition of claim 30, wherein the adenovirus of subgroup B is selected from the group of adenoviruses consisting of Ad16, Ad35, Ad11, and Ad51.
